(19) United States
(12) Patent Application Publication
(10) Pub. No.: US 2023/0036822 A1
(43) Pub. Date: Feb. 2, 2023

(54) IONTOPHORETIC WOUND TREATMENT DEVICE

(71) Applicant: HG Medical Technologies LLC, Fort Collins, CO (US)

(72) Inventor: Julian Henley, Fort Collins, CO (US)

(21) Appl. No.: 17/788,046

(22) PCT Filed: Dec. 24, 2020
(86) PCT No.: PCT/US20/67000

§ 371 Date: Jun. 22, 2022

Related U.S. Application Data
(60) Provisional application No. 62/954,142, filed on Dec. 27, 2019.

(51) Int. Cl. A61N 1/04

(52) U.S. Cl. CPC A61N 1/0468; A61N 1/0496; A61N 1/0428

(57) ABSTRACT

A wound treatment device includes a flexible substrate having a top side, a bottom side and an opening extending therebetween. A reservoir has a port in fluid communication with the opening, and an iontophoretic driver circuit is electrically coupled to a plurality of electrodes.

### CROSS-REFERENCE TO RELATED APPLICATIONS

This application claims priority to U.S. provisional application No. 62/954,142, filed Dec. 27, 2019, incorporated herein by reference in its entirety.

### BACKGROUND OF THE INVENTION

Various approaches and mechanisms for wound healing and management have been proposed over a long period of history. Today, the management of wounds (whatever their etiology) is mainly centered on prevention of fluid loss and bleeding from skin barrier compromise, prevention of host invasion by microorganisms and colonization of the wound, management of pain, and finally reconstruction or promotion of healing of the critical skin barrier. The current treatments are based on establishing a temporary barrier with polymers or bioactive membranes, control of microorganisms with systemic or topical antimicrobials, hemostasis and control of bleeding, management of pain, and ultimately tissue healing and tissue regeneration. There are many topical agents that have been shown to be beneficial to wound healing but their activity at site of injury is limited by lack of tissue penetration and thus systemic antibiotics and pain medication are used to help mitigate some of the difficulties with wounds.

Iontophoresis involves the application of an electromotive force to drive or repel oppositely charged ions through the dermal layers into the area to be treated; either into the surrounding tissues for localized treatment or into the circulatory system for systemic treatment. Positively charged ions are driven into the skin at the anode while negatively charged ions are driven into the skin at the cathode. One readily observed benefit of transdermal iontophoretic drug delivery is the increased efficacy of the drugs delivered in this fashion. Studies have shown increased skin penetration of drugs at anodic or cathodic electrodes regardless of the predominant molecular ionic charge. This effect is mediated by polarization and osmotic effects. Regardless of the electrical charge on the medicament employed, two electrodes are used in conjunction with the patient's skin to form a closed circuit to promote the penetration or absorption of the medicament through the skin underlying the working electrode.

Different approaches have been used to further improve the performance of iontophoretic devices. One approach is to use a multi-line dispersive electrode. For example, U.S. Pat. No. 5,160,316 issued to Henley and incorporated herein by reference describes the use of a multi-line dispersive electrode. Each line is driven by separate electronic circuits to assure wide dispersion and enhanced penetration of medicament. Such wide field electrodes can not only cover a wide area of the body without succumbing to “tunneling effects”, but they also provide sufficient skin penetration to function as a systemic drug delivery system. A second approach used to improve the performance of iontophoretic devices is to add ultrasonic elements to iontophoretic devices (this combination being referred to herein as ionosonic devices). For example, U.S. Pat. No. 5,658,247 issued to Henley and incorporated herein by reference describes a multi-line iontophoretic driver mounted on the application electrode with ultrasonic elements for enhanced intradermal delivery of therapeutic agents. Other improvements have been described for example in International Patent Application No. PCT/US2016/067450 address applications for wider areas and to prevent “current tunneling” effects and blistering. Multiple iontophoretically energized electrodes can be used such that each electrode is controlled by an independent current driver and limiter. Each electrode can overly a multiplicity of ports and can vary its voltage automatically until a specified flow current is reached within the area of its control and distribution. Advantageously, the accelerator device according to the various embodiments can be used for extended periods with externally applied treatment agents, and have fewer Ph-associated issues.

One particular area in need of improvement is wound healing and management related to burns. One of the challenges to the management of burns is the control of local site microorganism colonization and invasion as well as the repeated pain associated with surgical debridement (removal of dead tissue and eschar which fosters bacterial and fungal colonization). Such debridement removes the culture medium and prevents further skin damage by proliferating microorganisms and their toxins which further destroy any residual viable skin cells which would be critical to skin regeneration. It is not atypical for Level II burn to be converted to Level III because the few surviving skin cells are destroyed by invading microorganisms and their toxins. The other issue in management of burns is the management of wide area pain during the important wound debridement sessions that often require general anesthesia as local anesthesia fails to control the often-severe pain such multiple debridement procedures are associated with. Wound clinics strive to move some of this chronic wound management to outpatient management but management of wound pain remains challenging to outpatient management. As result there are many costly prolonged hospital admissions to manage slow healing wounds and control their pain and colonization by microorganisms.

Thus, what is needed in the art is a device and method that mitigates wound management issues by enhancing and amplifying the beneficial functions in wound management of already existing and well-established topical agents. Also needed in the art is a device and method that is particularly beneficial for healing and management of burn wounds.

### SUMMARY OF THE INVENTION

In one embodiment, a wound treatment device includes a flexible substrate having a top side, a bottom side and an opening extending therebetween, a reservoir having a port in fluid communication with the opening, and an iontophoretic driver circuit electrically coupled to a plurality of electrodes. In one embodiment, the plurality of electrodes are disposed on the bottom side of the substrate. In one embodiment, the plurality of electrodes comprises a plurality of active electrodes and a plurality of counter electrodes. In one embodiment, the plurality of active electrodes are disposed on a central portion of the substrate and the plurality of counter electrodes are disposed on the substrate outside the central portion. In one embodiment, the device includes an absorptive layer connected to the bottom side of the flexible substrate and in fluid communication with the opening and the reservoir. In one embodiment, the absorptive layer is a sponge layer disposed within gaps between a plurality of active electrodes. In one embodiment, the reservoir comprises a removable cap. In one embodiment, the reservoir comprises a trigger configured to activate the iontophoretic driver circuit upon insertion of a medicament agent. In one embodiment, the device includes a battery disposed on the substrate and electrically coupled to the iontophoretic driver circuit and the plurality of electrodes. In one embodiment, the iontophoretic driver circuit is a single channel iontophoretic driver circuit. In one embodiment, the iontophoretic driver circuit is a multichannel iontophoretic driver circuit. In one embodiment, the plurality of electrodes are configured for current polarization. In one embodiment, the device includes at least one of a skin adhesive flange and retaining strap. In one embodiment, the device includes an ultrasonic vibrational element attached to the substrate. A method of wound treatment includes the steps of depositing an anesthetic agent into the reservoir of the device and activating the plurality of electrodes to drive the anesthetic agent iontophoretically into a wound. A method of wound treatment includes the steps of depositing an antimicrobial agent into the reservoir of the device and activating the plurality of electrodes to drive the antimicrobial agent iontophoretically into a wound. A method of wound treatment includes the steps of depositing an hemostatic clotting promoting agent into the reservoir of the device and activating the plurality of electrodes to drive the hemostatic clotting promoting agent iontophoretically into a wound. A method of wound treatment includes the steps of depositing an vasoconstrictive agent into the reservoir of the device and activating the plurality of electrodes to drive the vasoconstrictive agent iontophoretically into a wound. A kit includes the device and a prepackaged agent configured to rupture upon insertion into the reservoir. The kit can include multiple prepackaged agents including two or more of an anesthetic agent, antimicrobial agent, hemostatic clotting promoting agent and a vasoconstrictive agent.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 is top view of a wound management device according to one embodiment.

FIG. 2 is a bottom view of the wound management device shown in FIG. 1 according to one embodiment.

FIG. 3 is a flow chart of a method of wound treatment according to one embodiment.

### DETAILED DESCRIPTION OF THE INVENTION

It is to be understood that the figures and descriptions of the present invention have been simplified to illustrate elements that are relevant for a more clear comprehension of the present invention, while eliminating, for the purpose of clarity, many other elements found in systems and methods of wound management. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Referring now in detail to the drawings, in which like reference numerals indicate like parts or elements throughout the several views, in various embodiments, presented herein is wound management device.

Described herein is a device and method that will modulate and improve the healing of wounds such as acute penetrating wounds, burn wounds, pressure induced wounds, and related avascular wounds, all of which have in common tissue exposure and skin barrier breakdown. The active bandage device described herein has the advantages of portability, disposability, sterility, can be deployed outside of point of care, has extended shelf life, and most important has an advanced therapeutic capability when activated at time of deployment. Both disposable and reusable configurations are described depending on clinical usage application in the field or at point of care.

Embodiments of the device described herein provides a powerful tissue penetration amplifier of topical medicaments that can be beneficial to the treatment and management of variety of wounds such as burns, penetrating wounds, and vascular etiology wounds such as pressure sores. This minimizes the shortcomings of current technology for the treatment of wounds, which can be difficult, expensive, painful to the patient, and ultimately devastating to their lives. The devices described henceforth have a significant benefit to the effective treatment of acute and chronic wounds by virtue of their amplified penetration of active medicaments directly into the affected tissues in concentrations that topical wound application cannot achieve without this technology.

It is the central approach of the embodiments described herein to mitigate wound management issues by enhancing and amplifying the beneficial functions in wound management of already existing and well-established topical agents. The device wound bandage described herein has all the benefits of existing protective bandages but also has the ability to drive into the wound itself medicaments when such are introduced into the bandage reservoir. Agents for antimicrobial, analgesic, vasoconstrictive and hemostatic properties can now be readily applied and penetrated into the active wound.

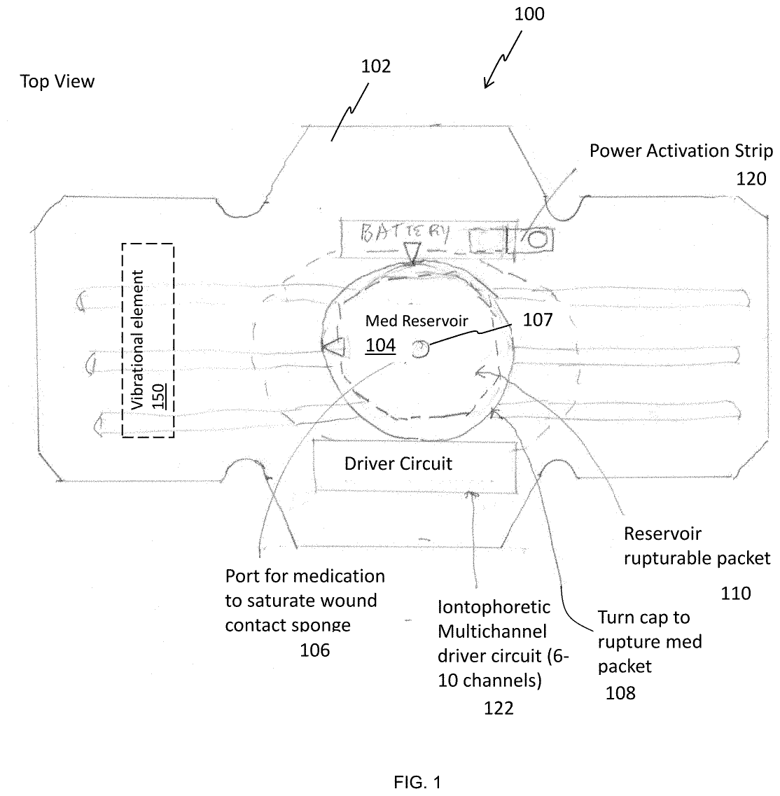

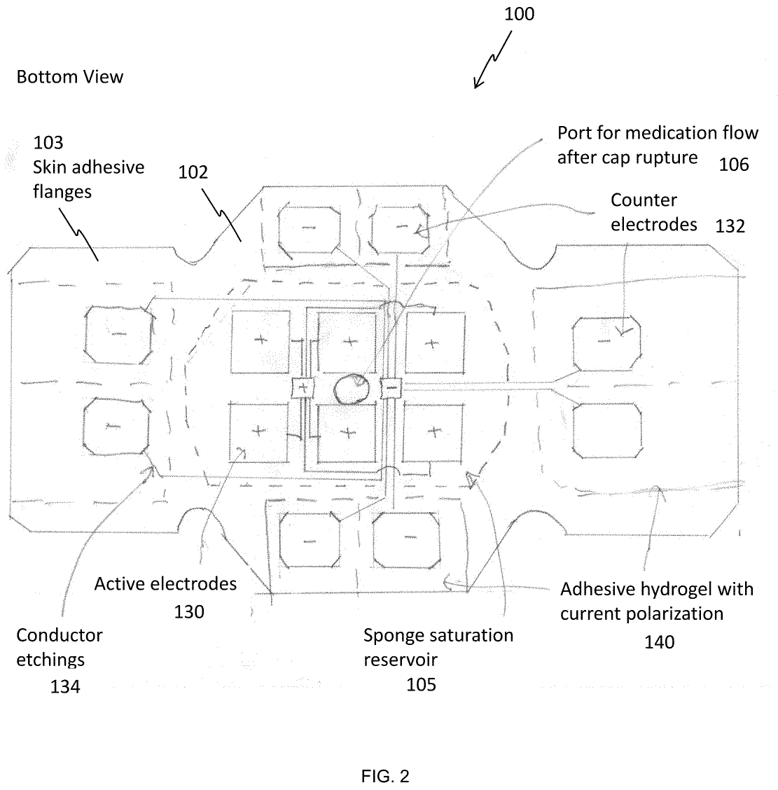

With reference now to FIGS. 1 and 2, a top (FIG. 1) and bottom (FIG. 2) view of a wound management device 100 is shown according to one embodiment. The device 10 is built on a flexible substrate 102 having a top side, a bottom side and an opening 107 extending therebetween. The reservoir 104 includes a port 106 in fluid communication with the opening 107. An iontophoretic driver circuit 122 is electrically coupled to a plurality of active electrodes 130 and counter electrodes 132. In one embodiment, the electrodes 130, 132 are disposed on the bottom side of the substrate 102. In one embodiment, the active electrodes 130 are disposed on a central portion of the substrate 102 and the counter electrodes 132 are disposed on the substrate 102 outside the central portion at the flanges 103. Electrode circuits can be connected via conductor etchings 134. Hydrogel can be implemented with current polarization 140. In one embodiment, the device 100 includes an absorptive layer connected to the bottom side of the flexible substrate 102 and in fluid communication with the opening 106 and the reservoir 104. The absorptive layer can be a sponge layer 105 disposed within gaps between a plurality of active electrodes 130. In one embodiment, the reservoir 104 includes a removable cap. In one embodiment, the reservoir 104 includes a trigger configured to activate the iontophoretic driver circuit 122 upon insertion of a medicament agent. In one embodiment, a battery is disposed on the substrate 102 and electrically coupled to the iontophoretic driver circuit 122 and the electrodes 130, 132. In one embodiment, the iontophoretic driver circuit 122 is a single channel iontophoretic driver circuit. In one embodiment, the iontophoretic driver circuit 122 is a multichannel iontophoretic driver circuit. In one embodiment, the electrodes 130, 132 are configured for current polarization. In one embodiment, the device 100 includes at least one of a skin adhesive flange 103 and a retaining strap. In one embodiment, an ultrasonic vibrational element 150 is attached to the substrate 102. A kit including the device and a prepackaged agent can be configured to rupture upon insertion into the reservoir.

Thus in one aspect, a wound dispersive wound application electrode and driver device can be used to rapidly infuse topical anesthetic agent directly into the burn wound to achieve rapid pain control just prior to mechanical debridement. A similar electrode and device can be used to drive an antimicrobial medicament into the wound and or eschar to maintain control of colonization between treatments and manage many more patients on outpatient basis during the extended wound healing period. Such device and electrodes will be helpful in the management of the burned patient within the hospital as well as outpatient basis.

In another embodiment, an acute penetrating wound device-bandage is described that not only occludes the open wound, like a bandage, but also during the application period (e.g. when the injured individual is being transported or evacuated) the device continually drives into the open wound. An antimicrobial agent, an analgesic/anesthetic agent, a vasoconstrictive agent, and/or clotting agent are applied by the device to minimize bleeding. Such agents can be driven all concurrently in combination or individually depending on the circumstances. The design, construction, packaging, deployment, and activation of its incorporated therapeutic electrokinetic drive is described in embodiments herein. These embodiments of an active electrokinetic wound device and bandage offers significant benefits to current wound management technologies. For example, it can keep contaminating microorganisms at bay, control the pain without systemic obtunding morphine or the like, and help control active bleeding, offering a significant advantage in the treatment and management of acute penetrating injuries, burn wounds, and chronic vascular profusion wounds.

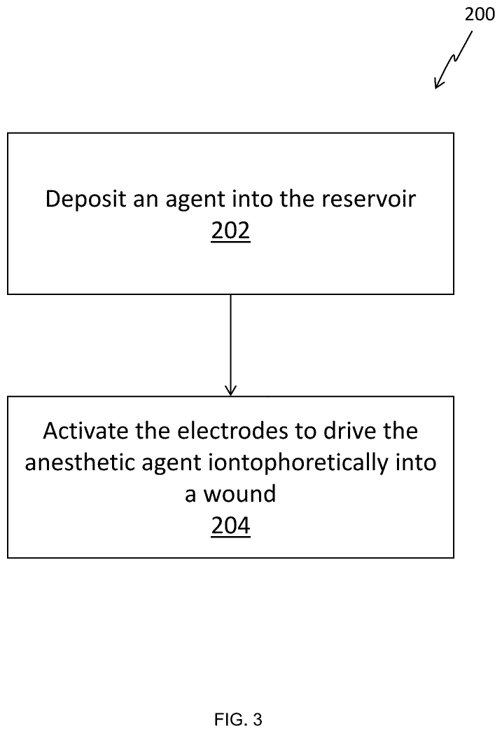

A method 200 of administering various agents are also disclosed, with reference now to FIG. 3 according to one embodiment. The method 200 includes the steps of depositing an agent into the reservoir 202 and activating the electrodes to drive the agent iontophoretically into a wound 204. The in certain embodiments may be an anesthetic agent, an antimicrobial agent, a hemostatic clotting promoting agent, and a vasoconstrictive agent.

Embodiments of the device and method described herein are intended for the treatment of open wounds which involves the breakdown of an interposing skin barrier between the outside world and internal tissues. Previously described electrokinetic and iontosonic devices were intended to overcome such skin barrier by using ported electrical current to carry medication into mucosa or skin. In embodiments of the instant invention, the skin barrier is already compromised, so the process that can electrically overcome the skin barrier and deliver medicaments into the underlying tissue lends itself to directly deliver medicaments to the underlying tissues in wound where the natural barrier to such penetration is already compromised. Incorporating the electrokinetic delivery system with or without the ultrasonic elements into a wound bandage device will greatly enhance the penetration of medicaments into underlying wound tissues if such medication is interposed into the absorbent layer between the conductive dispersive electrodes and the open exposed tissue of the wound. The design of this novel active wound bandage incorporates electrokinetic and iontosonic device technology to improve the treatment efficacy of open wounds by amplifying the in-situ penetration of healing agents whose activity has always been limited by lack of tissue penetration. This active penetration enhanced bandage device provides a novel solution to the age old problem of enhancing the wound healing process. By judicious selection of medicaments that will be introduced into this “active bandage device” novel benefits can be derived such as control of local pain with use of local topical anesthetic agents, control of microbial proliferation and further tissue damage through insertion of antimicrobials and or silver based compounds/ions, and finally and equally important control of bleeding with use of vasoconstrictive agents and clotting promoting agents.

Multichannel iontophoretic dispersive electrodes are incorporated within the portion of the active bandage that also contains the absorptive layer that retains the medicament in the interposed space between the electrode and the open exposed tissue. Because the resistive barrier of the damage skin is greatly reduced it is clear that lower voltage and power requirements can be utilized on the driver circuitry that likewise can be miniaturized and incorporated into the active bandage. Each electrode channel will have current limited feature as described previously and preferably not exceed 2 ma/cm2 flux energy. Higher current fluxes can be utilized when needed especially in situations where conductive barrier is compromised, resistive load is lower and need for more delivery of medicament in less time is desired.

To mitigate any medication degradation from contact with conductive elements (electrodes) it can be beneficial to encapsulate the unit dose medicament within an inert capsule/bubble that will then be ruptured at time of bandage deployment and saturate the interposed absorptive layer which will remain in direct contact with damaged skin and underlying tissue. Another feature of the device that can be prepackaged sterile is the fact that only at time of deployment will the embedded electronic driver that controls the current to each channel will be powered and activated.

As discussed above, piezoelectric elements in proximity of the dispersive iontophoretic channels can be incorporated. These elements will require lower energy levels than the ones used conventionally because the wound is open in this case and in prior applications the ultrasonic elements were used to break down the skin barrier by enhancing its porosity. So in this case, the piezoelectric elements only aid in the electromechanical dispersion as well as supplant the tissue penetration of the medicaments applied within the active wound bandage. The active wound bandage device may function well as an electro kinetic device when using electrokinetic electrodes alone or in combination with piezoelectric elements which will than function better in some circumstances as an iontosonic or ionosonic device. The two technologies described herein can be used as stand-alone modality or as combination of both used concurrently within the same bandage device. Either form (electrokinetic or iontosonic) offers benefits.

Yet another feature of the embodiments described herein is the incorporation of current polarization (see e.g. U.S. Provisional Application No. 62/778,410, filed Dec. 12, 2018). This feature greatly enhances the performance and safety of the multichannel design. The features and construction of a ported multichannel iontophoretic dispersive have can be incorporated in this device as well according to the designs previously described.

There are variations of size, shape, materials and electro distributions that could accommodate a variety of anatomical body applications. The particular embodiment shown in the figures has not only the ability to act as a wound dressing but also is active in terms of infusing the wound with an anesthetic agent anticlotting agent antimicrobial agent and possibly even vasoconstrictive agent. All such agents can be encapsulated in their own rupture bowl units, for example in a mylar bag because of its sturdiness and its chemical inertness. Such retaining units such as mylar baggie containing active agents will be supplied concurrently or inserted into the bandage reservoir at the time of activation. One advantage for separate encapsulation is because of the inert body of the mylar and this therefore it does not require shelf-life studies on the activation of such agents when they are fully embedded in a dressing material. In other words, at the time of application the bag is ruptured and permeates into the portion of the wound dressing that's in direct contact with the open wound. Supplying active pharmaceutical agents as a separate unit dose that is applied at the time of activation is a preferred methodology from the standpoint of sterility and long-term activity. Sterilization techniques that will be used to sterilize the application bandage will not affect the activity of the pharmaceutical agents because the supplied pharmaceutical agents will be are supplied separately encapsulated in the mylar baggie.

Upon insertion into the medication compartment of the bandage the baggie is ruptured and the content is then diffused into the absorptive portion of the bandage itself that's interposed between the wounds and the active electrode overlying the pores of the retention layer. This retention layer can be a polymer with an open cell structure preferably hydrophilic as it rapidly then absorbs through capillary action and distributes the ruptured content of the mylar baggie throughout the layer that is now in direct contact with the wound.

It is also intended in certain embodiments that at the time of insertion of the baggie into the bandage compartment a thin mylar sheet is removed from the battery contact which then allows activation of the electronics which in turn will create a multichannel iontophoretic drive that we often refer to as electrokinetic technology into the surface contact of the open wound. Iontophoretic technology has always had a limitation of current flocks and has a limited ability to cover a surface area. However, by introducing multiple channels a much improved a more controlled distribution of the current is achieved, which in turn is carried by the pharmaceutical agents embedded through capillary action in the interposed absorptive layer that's now in direct contact with the open wound.

The counter electrode can be distributed outside of the open wound in contact with skin within interposed hydrogel adherent and conductive layer. Those skilled in the art will be familiar with conductive gel adhesion and conductive ability. Both the active wound electrode and the counter electrode can be embedded in the flanges 103 of the adherent bandage. Current polarization can prevent inter-channel shorting.

Yet another desirable ergonomic feature of embodiments of the bandage is the retaining elastomer of sufficient length to be able to come around an adult chest with a loop and Velcro and retention at the end. This elastomer ribbon will help with the patients that have e.g. a hairy chest and substandard direct adhesion of the Hydrogel that's in contact with the with the skin. This elastomer can also function to offer additional pressure on the wound. Small venules can be compressed and they clotting and an agent within the mylar baggie will facilitate formation of hemostasis.

Embodiments of the bandage will usually serve between 6 to 8 distribution channels and the electronics that drives them will be embedded in the bandage itself. The design of the electronics that the controls the current flow through the electrode has been well described in the prior art and can be incorporated onto a disposable bandage. The bandage is therefore constructed of a multichannel iontophoretic driver and interposed absorptive layer that take the medication from the reservoir and likewise interposes the medications to be driven into the open wound by the iontophoretic drive. This absorptive layer will be saturated with active pharmaceutical agents or agents that are inserted into the outer reservoir by capillary diffusion.

In one embodiments, the bandage is sufficient to treat a wound size in the range of 20-30 cm\hat{ }2. In certain embodiments, upon placement of the device to occlude the wound the operator will turn the cap like reservoir clockwise which in turn will rapture and internal mylar baggie filled with suitable medication combination (discussed previously) which in turn will saturate the absorptive layer in contact with the open wound. The next step is to remove the plastic ribbon that now activates the power supply by removing interposed layer from battery compartment and activates the functionality of the iontophoretic driver electronics which in turn energize the dispersive electrodes to directly drive into the open wound the desired medicaments.

This preferred embodiment has the advantages that the device can be packaged sterile and the medication baggie has established shelf life as it remains in contact with well-established polymer container shelf life studies of molecular stability over time. The incorporated battery already has well established shelf life data. As such, this device will present manageable regulatory challenges and will be able to be deployed in the area of open wound management. Incorporation of ultrasonic elements into the design of this device as described in prior patents may offer additional wound management benefits especially in areas of medication dispersion, wound debridement, and wound penetration.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

What is claimed is:

1. A wound treatment device comprising:
a flexible substrate having a top side, a bottom side and an opening extending therebetween;
a reservoir comprising a port in fluid communication with the opening; and
an iontophoretic driver circuit electrically coupled to a plurality of electrodes.

2. The device of claim 1, wherein the plurality of electrodes are disposed on the bottom side of the substrate.

3. The device of claim 1, wherein the plurality of electrodes comprises a plurality of active electrodes and a plurality of counter electrodes.

4. The device of claim 3, wherein the plurality of active electrodes are disposed on a central portion of the substrate and the plurality of counter electrodes are disposed on the substrate outside the central portion.

5. The device of claim 1 further comprising:
an absorptive layer connected to the bottom side of the flexible substrate and in fluid communication with the opening and the reservoir.

6. The device of claim 5, wherein the absorptive layer is a sponge layer disposed within gaps between a plurality of active electrodes.

7. The device of claim 1, wherein the reservoir comprises a removable cap.

8. The device of claim 1, wherein the reservoir comprises a trigger configured to activate the iontophoretic driver circuit upon insertion of a medicament agent.

9. The device of claim 1 further comprising:
a battery disposed on the substrate and electrically coupled to the iontophoretic driver circuit and the plurality of electrodes.

10. The device of claim 1, wherein the iontophoretic driver circuit is a single channel iontophoretic driver circuit.

11. The device of claim 1, wherein the iontophoretic driver circuit is a multichannel iontophoretic driver circuit.

12. The device of claim 1, wherein the plurality of electrodes are configured for current polarization.

13. The device of claim 1 further comprising:
at least one of a skin adhesive flange and retaining strap.

14. The device of claim 1 further comprising:
an ultrasonic vibrational element attached to the substrate.

15. A method of wound treatment comprising:
depositing an anesthetic agent into the reservoir of the device of claim 1;
activating the plurality of electrodes to drive the anesthetic agent iontophoretically into a wound.

16. A method of wound treatment comprising:
depositing an antimicrobial agent into the reservoir of the device of claim 1;
activating the plurality of electrodes to drive the antimicrobial agent iontophoretically into a wound.

17. A method of wound treatment comprising:
depositing an hemostatic clotting promoting agent into the reservoir of the device of claim 1;
activating the plurality of electrodes to drive the hemostatic clotting promoting agent iontophoretically into a wound.

18. A method of wound treatment comprising:
depositing an vasoconstrictive agent into the reservoir of the device of claim 1;
activating the plurality of electrodes to drive the vasoconstrictive agent iontophoretically into a wound.

19. A kit comprising the device of claim 1 and a prepackaged agent configured to rupture upon insertion into the reservoir.

20. The kit of claim 19 further comprising:
a plurality of prepackaged agents including two or more of an anesthetic agent, antimicrobial agent, hemostatic clotting promoting agent and a vasoconstrictive agent.
